(19) United States
(12) Patent Application Publication
(10) Pub. No.: US 2023/0280320 A1
(43) Pub. Date: Sep. 7, 2023

(54) SYSTEM AND METHOD OF CALIBRATING SENSING INSTRUMENTS

(71) Applicant: THE ARMSTRONG MONITORING CORPORATION, Ottawa (CA)

(72) Inventors: Andrew COOPER, Ottawa (CA); Joanne JOHNSON, Ottawa (CA); Stephanie PICHETTE, Ottawa (CA); Rodney SPRULES, Ottawa (CA); Curtis TOUPIN, Ottawa (CA); Aaron WATKINS, Ottawa (CA)

(21) Appl. No.: 18/008,346

(22) PCT Filed: Jun. 4, 2021
(86) PCT No.: PCT/CA2021/050773

§ 371 Date: Dec. 5, 2022

Related U.S. Application Data
(60) Provisional application No. 63/035,318, filed on Jun. 5, 2020.

(51) Int. Cl. G01N 33/00

(52) U.S. Cl. CPC G01N 33/0006

(57) ABSTRACT

A calibration system and method for calibrating an instrument are provided. The system comprises at least one sensor, a processor, and a memory comprising instructions which, when executed by the processor, configure the processor to perform the method. The method comprises obtaining a series of sensor readings, determining variations between changes in successive (or near successive) sensor readings from the series of sensor readings, estimating a stabilization point of the sensor readings by identifying at least one gas sensor reading from series of sensor readings at which the change in sensor reading from at least one previous sensor readings is small relative to the variations, and adjusting a parameter in the instrument that represents an association between sensor readings and a known physical quantity based on the stabilization point.

### CROSS REFERENCE TO RELATED APPLICATIONS

This application is a non-provisional of, and claims all benefit, including priority, to U.S. Application No. 63/035,318, dated Jun. 5, 2020 entitled SYSTEM AND METHOD OF CALIBRATING GAS SENSING INSTRUMENTS, which is incorporated herein in its entirety by reference.

### FIELD

The present disclosure relates generally to systems and methods of calibrating sensing instruments.

### BACKGROUND

Gas sensors degrade in terms of signal output and drift through time and therefore should be calibrated regularly. Two key challenges are often encountered in doing this calibration. First, the sensor response time and signal output vary depending on the environment within which they operate; i.e., they are affected by temperature, humidity, radio frequency (RF), the presence of other gases, etc. Second, different service technicians might wait different (and insufficient or excessive) amounts of time to enable the sensor to reach the output level required for calibration.

There are two methods that are commonly used in sensor calibration. One common calibration method involves waiting until the maximum output (or minimum output in the case of a sensor with reverse polarity) is achieved and calibrating the sensor (i.e., adjusting the gain) accordingly. The problem with this is that it takes a long time. There are many associated problems with this waiting period, given that the signal approaches a maximum asymptotically. First, the technician might not wait long enough. Additionally, the longer it takes to wait for maximum output, the more toxic or combustible gas that is consumed, which is expensive in terms of both labour and gas consumption and which results in prolonged release of toxic or combustible gases into the air, with a potential negative impact on the environment. Another potential calibration method is determining the T80 or T90, defined as the time at which the sensor's response to the gas is 80% or 90% of the final response, respectively, which involves the exposure of the sensor to gas until it reaches 80% or 90% of its maximum output, and extrapolates the necessary gain. Consequently, while this method is much faster than waiting until the maximum output is achieved, the results are much less accurate and may lead to over-reporting and costly false alarms.

Calibration routines have also been developed based on analysis of when the change in readings becomes small, relative to some pre-defined threshold. However, because both the speed and magnitude of sensor response is impacted by a variety of factors, a different threshold would be optimal for every environment (i.e., every possible permutation of temperature, humidity, pressure, presence of other gases, etc.), which is not practical. Consequently, the use of the change relative to some pre-defined threshold would be lower than optimal for some situations and higher than optimal for other situations.

Because sensor output is impacted by temperature, humidity and other factors, the maximum (or minimum) expected output, as well as the length of time necessary to reach those, also varies by those factors. Lookup tables are frequently created for temperature or humidity, based on specifications provided by sensor manufacturers, but manufacturers generally do not provide output data by varying combinations of temperature and humidity. It is possible to conduct research and gather data for numerous combinations of temperature and humidity points, but this is very time consuming and still subject to human error.

### SUMMARY

In some embodiments, there is provided a calibration system for calibrating an electronic instrument. The calibration system comprises at least a sensor, a processor and a memory comprising instructions which when executed by the processor configure the processor to obtain a series of sensor readings, determine variations between successive (or near successive) sensor readings, estimate a stabilization point of the sensor readings by identifying at least one sensor reading from a series of sensor readings at which the change in sensor reading from at least one previous sensor reading is small relative to the variations, and adjust a parameter in the instrument that represents an association between sensor readings and a known physical quantity based on the stabilization point.

In some embodiments, there is provided another calibration system for calibrating an electronic instrument. The calibration system comprises at least one gas sensor, a processor, and a memory comprising instructions which when executed by the processor configure the processor to obtain a series of gas sensor readings, determine variations between changes in successive gas sensor readings from the series of gas sensor readings, estimate a stabilization point of the gas sensor readings by identifying at least one gas sensor reading from series of gas sensor readings at which the change in sensor reading from at least one previous gas sensor readings is small relative to the variations, and adjust a parameter in the instrument that represents an association between sensor readings and a known physical quantity of gas based on the stabilization point.

In some embodiments the calibration system comprises a processor, and a memory comprising instructions as well as a temperature sensor, and/or a humidity sensor, and/or a pressure sensor and/or a vibration sensor and/or a motion sensor and/or a light and/or a sound sensor and/or a particle sensor. In these embodiments, as described in the ones above, the calibration system obtains a series of sensor readings, determine variations between changes in successive sensor readings from the series of sensor readings, estimates a stabilization point of the sensor readings by identifying at least one sensor reading from a series of sensor readings at which the change in sensor reading from at least one previous sensor reading is small relative to the variations, and adjusts a parameter in the instrument that represents an association between sensor readings and a known physical quantity based on the stabilization point.

In some embodiments, there is provided a method for calibrating an instrument. The method comprises obtaining a series of sensor readings, determining variations between changes in successive sensor readings from the series of sensor readings, estimating a stabilization point of the sensor readings by identifying at least one sensor reading from series of sensor readings at which the change in sensor reading from at least one previous sensor readings is small relative to the variations, and adjusting a parameter in the instrument that represents an association between sensor readings and a known physical quantity based on the stabilization point.

In some embodiments, there is provided a method for calibrating an instrument. The method comprises obtaining a series of gas sensor readings, determining variations between changes in successive gas sensor readings from the series of gas sensor readings, estimating a stabilization point of the gas sensor readings by identifying at least one gas sensor reading from series of gas sensor readings at which the change in sensor reading from at least one previous gas sensor readings is small relative to the variations, and adjusting a parameter in the instrument that represents an association between sensor readings and a known physical quantity of gas based on the stabilization point.

In various further aspects, the disclosure provides corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the instant disclosure.

### DESCRIPTION OF THE FIGURES

Embodiments will be described, by way of example only, with reference to the attached figures, wherein in the figures:

FIG. 1 illustrates, in a schematic, an example of a calibration system for calibrating an instrument, in accordance with some embodiments;

FIG. 2 illustrates, in a flowchart, an example of a method of calibrating an instrument, in accordance with some embodiments;

FIG. 3 illustrates, in a flowchart, an example of a method of calibrating a gas sensor, in accordance with some embodiments;

FIG. 4 illustrates, in a flowchart, an example of a method of gathering the buffering data and receiving input, in accordance with some embodiments;

FIG. 5 illustrates, in a flowchart, an example of a method of finding a zero, in accordance with some embodiments;

FIG. 6 illustrates, in a flowchart, an example of a method of waiting for gas (or other target phenomena), in accordance with some embodiments;

FIG. 7 illustrates, in a flowchart, an example of a method of determining the span, in accordance with some embodiments;

FIG. 8 illustrates, in a flowchart, an example of a method of determining stability, in accordance with some embodiments;

FIG. 9A illustrates, in a graph, an example of time versus sensor responses for prior methods using T80/90;

FIG. 96 illustrates, in a graph, an example of time versus sensor responses for prior methods using Maximum Output;

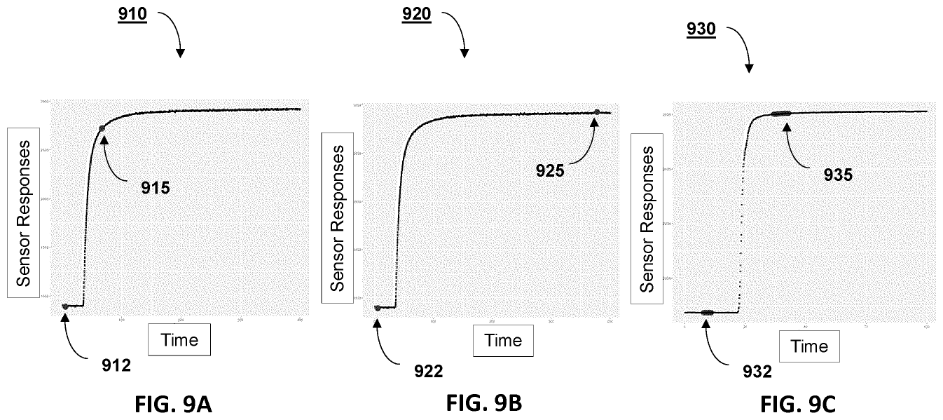

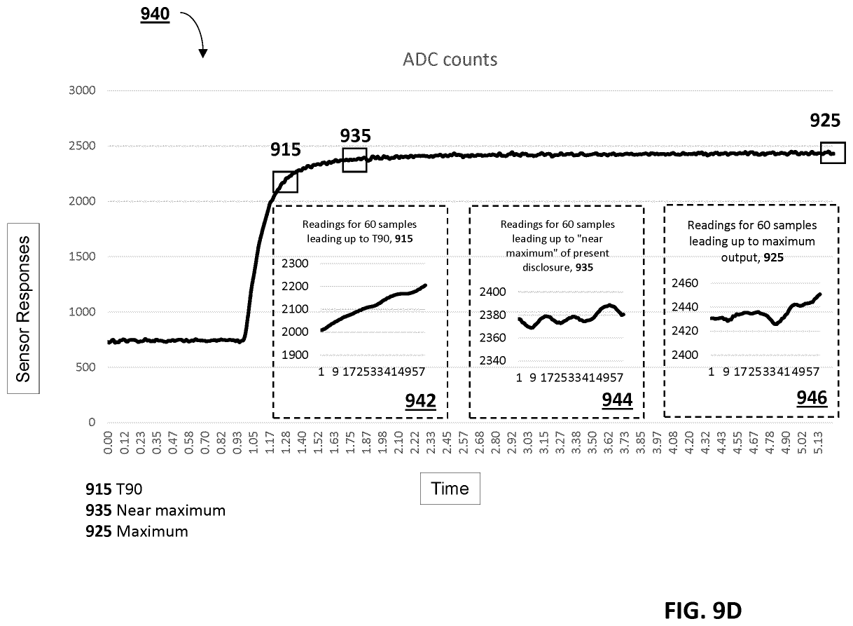

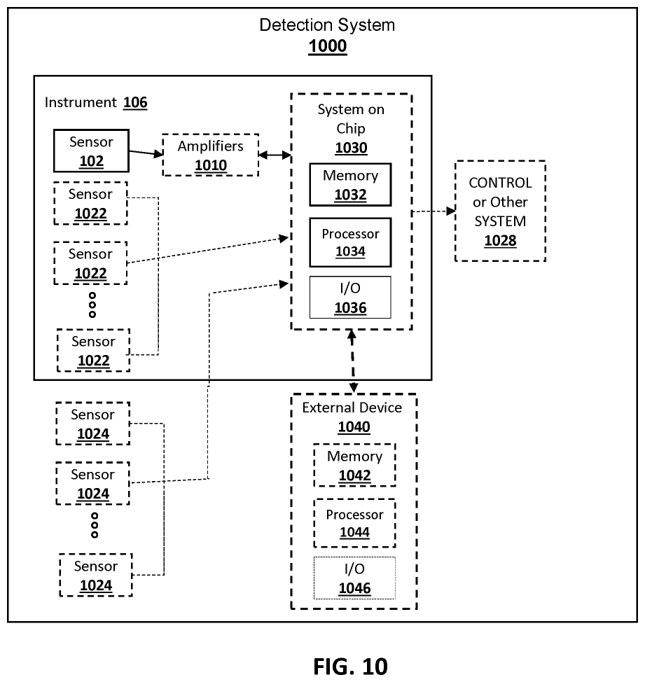

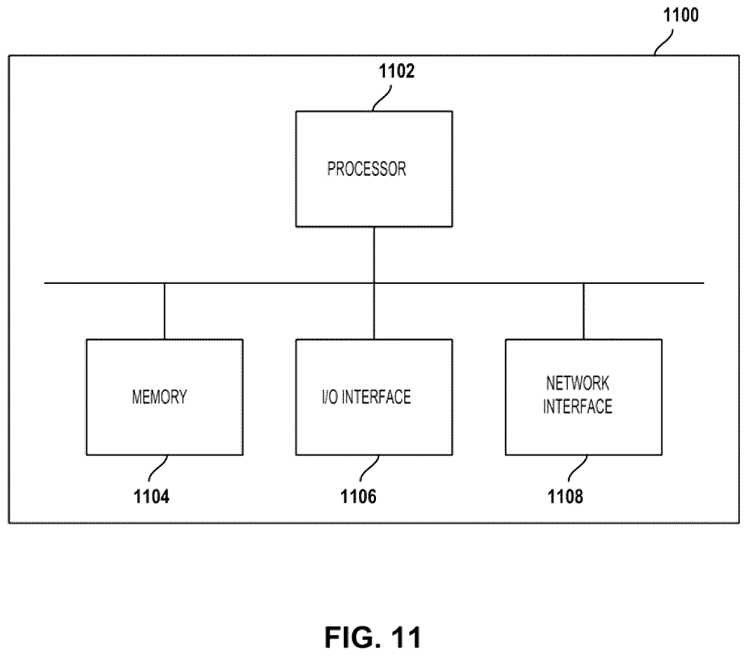

FIG. 9C illustrates, in a graph, an example of time versus sensor responses for the method described herein, in accordance with some embodiments;

FIG. 9D illustrates, in a graph, the examples of sensor response versus time for all three methods of FIGS. 9A to 9C;

FIG. 10 illustrates, in a schematic, a detection system which reacts to a signal or stimulus from a physical phenomenon, in accordance with some embodiments;

FIG. 11 is a schematic diagram of a computing device such as a server; and

FIG. 12 shows a schematic representation of the system chip, as a combination of software and hardware components in a computing device.

It is understood that throughout the description and figures, like features are identified by like reference numerals.

### DETAILED DESCRIPTION OF THE EMBODIMENTS

One objective of the present disclosure is to develop a method of calibrating sensing instrumentation (such as a gas sensor, a temperature sensor, a humidity sensor, a pressure sensor, a vibration sensor, a motion sensor, a light sensor, a sound sensor, a particle sensor, a biosensor and/or any sensor in an electronic detection or sensing instrument) that is:
Fast
Precise and consistent
Automatically adjusts to drive gassing, or application of other target phenomena for the appropriate amount of time
Gathers and leverages data on factors that might impact gas (or other phenomena) readings, such as temperature, humidity, vibration, pressure, etc.
Requires little computational resources, data storage and/or power

It is also worth noting that the smaller the data storage and power requirement, the more feasible it is do the calibration on the site where a phenomena (e.g., gas) is being detected.

In some embodiments, variations (e.g., small “random” variations) inherent in the signal are used as a way to self-assess when the sensor has reached an output level that is close enough to its maximum (or minimum in the case of a sensor which produces a decreasing signal) that it can be used to calculate the adjustment required for calibration. For example, an initial reference point (for example the signal at a zero gas concentration) and the “near” maximum at a non-zero gas concentration point are estimated by finding the output where the change in output (i.e., the slope of the signal) is sufficiently close to zero (i.e., “near” zero) over a given set of samples. As another example, the initial reference point and the “near” maximum might pertain to temperature in degrees centigrade, relative humidity measurements, vibration (rate of change in displacement per unit time), pressure (Pascal), frequency of intensity of light or sound waves, the number of particles, etc.

It should be understood that the “random” variations in the signal are the variations generally present in all electronic signals. They are sometimes referred to as electrical noise, or harmonics. They represent “apparently” random variations that typically, but may not, have a somewhat repeatable or consistent amplitude or frequency superimposed onto the signal. In many cases these variations are not random, but influenced by electrical interference, fluctuating power, degradation in the equipment (e.g. inconsistent distribution of electrolytes in an electrochemical sensor) and/or other factors and in other cases there may be no apparent or detectable reason for the variation. A person skilled in the art will understand that a finite impulse response (FIR) filter may be used to ensure that there is enough, but not too much variation around the signal trend, and/or to normalize the variations in the signal.

It should be understood that the terms ‘near maximum’ or ‘near zero’ include readings that are approximately near the actual maximum reading or zero reading, respectively. In some embodiments, the near maximum or near zero may include the actual maximum or actual zero reading, respectively. It should also be understood that the ‘near maximum’ or ‘near minimum’ may pertain to the first or second difference of the change (i.e., where the slope begins to increase, stabilize, or hit an inflection point or some other pattern or range of response). It should also be understood that references to approximate limits to gas (or other phenomena) readings (i.e., near maximum, near minimum, near zero) in this disclosure may be different for different gases (or other phenomena being measured). It should also be understood that throughout this disclosure, references to the terms ‘maximum’, ‘minimum’ and ‘zero’ include ‘near maximum’, ‘near minimum’ and ‘near zero’.

In some embodiments, the outputs at any two known reference values are used by finding the output where the slope of the signal, or the change in the slope, is sufficiently close to approximately zero (or some other threshold), relative to small “random” variations inherent in the signal. In some embodiments, calibrations may be performed with a range different than the minimum and the maximum. For example, this approach would apply with an oxygen sensor whose first reference is the background concentration and secondary reference is 0% volume. This approach might also apply to using the start of gassing and the inflection point as reference points.

In another embodiment, the standard deviations can be used as a measure of the small random variations inherent in the signal, and the point at which the slope (or slope changes) are smaller than some number of standard deviations can be used to identify stability.

In another embodiment, the point at which the slope is less than the standard deviation of the signal can be approximated by identifying the point (and associated output) at which “near stability” has been achieved. In one embodiment, this point is found where the proportion of readings in which the first difference between any two output readings over a pre-defined latter set of readings is consecutively less than half the maximum first difference observed in a pre-defined earlier set of readings, for a predefined minimum number of periods. This is just one example of how stability can be measured using the small random variations inherent in the signal, and there are other algorithms or approaches that could be taken.

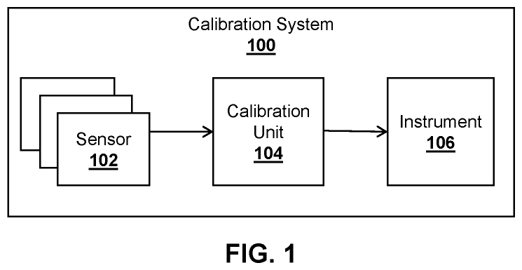

FIG. 1 illustrates, in a schematic, an example of a calibration system 100 for calibrating an instrument, in accordance with some embodiments. The system 100 comprises at least one sensor 102, a calibration unit 104, and an instrument 106. In some embodiments, the at least one sensor 102 may be one or more gas sensors, temperature sensors, humidity sensors, pressure sensors, vibration sensors, motion sensors, light sensors, sound sensors, and/or particle sensors. Other components may be added to the system 100, including one or more amplifiers. As will be described in more detail below, the calibration unit 104 receives sensor readings from the sensor 102 and adjusts or calibrates a parameter in the instrument 106. For example, the received sensor readings may be gas sensor readings in the case where the at least one sensor 102 is a gas sensor. In some embodiments, the sensor 102 and/or the calibration unit 104 may be a component of instrument 106. Other components may be added to the system 100, including one or more amplifiers.

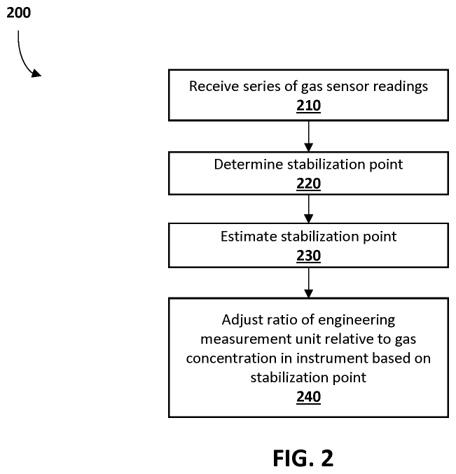

FIG. 2 illustrates, in a flowchart, an example of a method 200 of calibrating an instrument, in accordance with some embodiments. The method 200 may be performed by the calibration unit 104 or an instrument 106 that includes logic performed by the calibration unit 104. The method 200 includes obtaining 210 a series of sensor readings. I.e., the calibration unit 104 logic receives readings from the sensor 102 and/or instructs a device or instrument having a sensor 102 to obtain the reading. Small “random” variations between changes in successive sensor readings from the series of sensor readings may then be determined 220. A stabilization point of the sensor readings may then be estimated 230 by identifying at least one sensor reading from the series of sensor readings at which the change in sensor reading from at least one previous sensor reading is small relative to the small “random” variations. In some embodiments, the stabilization point may comprise a point where the change in the sensor reading reaches zero or near zero. Once the stabilization point is estimated 230, a parameter representing an association between sensor readings and a known physical quantity (in some embodiments, a target value may represent the physical quantity) in the instrument is adjusted 240. In some embodiments, this comprises adjusting, in the instrument 106, a ratio of an engineering measurement unit relative to the known physical quantity. In some embodiments this ratio may be adjusted in the firmware. In other embodiments, this ratio may be adjusted by changing the physical gain in one or more amplifiers. In still other embodiments, this ratio may be adjusted through a combination of firmware and physical gain adjustments of one or more amplifiers. Other steps may be added to the method 200.

In some embodiments, the sensor readings in FIG. 2 may be sensors 102 and sensor readings pertain to temperature sensors, humidity sensors, pressure sensors, vibration sensors, motion sensors, light sensors, sound sensors, and/or particle sensors. The physical quantity in the association being adjusted is relative to the type of sensor 102. For example, for a gas sensor, the physical quantity is a gas concentration level.

In some embodiments, the system 100 may comprise, and method 200 may apply to, more than one different type of sensor 102. In such embodiments, each type of sensor 102 may obtain separate measurements and stored in different memory files. The method 200 steps may be applied separately to those separate measurements independent from the other measurements. The system 100 may be configured to calibrate one parameter pertaining to one type of sensor at a time, or different parameters for different sensors in parallel (but separate) applications of the method 200.

The remaining methods will be described for gas sensors for ease of presentation. However, it should be understood that the following methods may also apply to different types of sensors with appropriate modifications. For example, the phenomenon being measured and the physical quantity being assessed can be replaced with that which applies to the different type of sensor. I.e., references to gas sensors or measurements or other readings pertaining to gas sensors may be replaced, mutatis mutandis, with those that apply to a different type of sensor (whether or not this is explicitly indicated below).

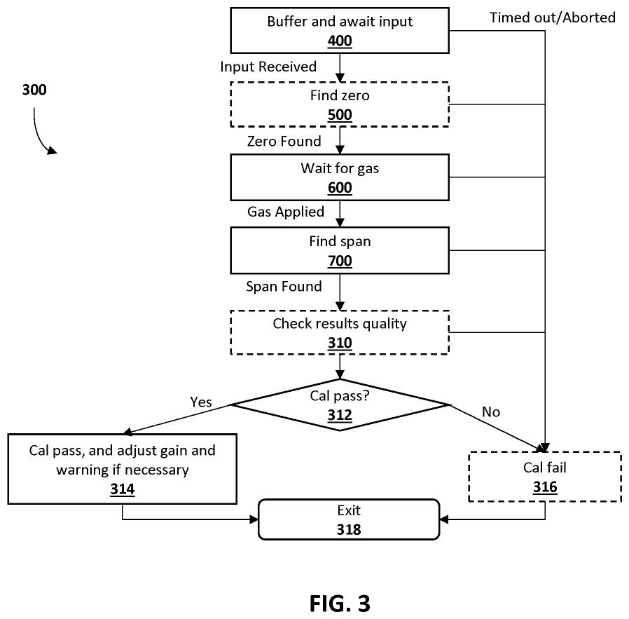

FIG. 3 illustrates, in a flowchart, another example of a method 300 of calibrating an instrument, in accordance with some embodiments. The method 300 may be performed by the calibration unit 104 or an instrument 106 that includes logic performed by the calibration unit 104. FIG. 3 shows the high-level steps (described in greater detail below) involved in calibrating a gas sensing instrument 106 using small random variations inherent in the signal. The method 300 comprises gathering sample data in a buffer and receiving input 400, (optionally) determining a zero output level 500, waiting for gas from a physical system (or other physical quantity) to be applied 600, and determining the signal span 700. Optionally, the quality of the sensor output may be checked 310. Once the span is determined 700 (or the sensor output is checked 310), if the calibration passed 312, then the instrument is adjusted 314. Otherwise 312, the calibration failed and the calibration mode is exited 316. It should be understood that a calibration can pass or fail. In order to “pass” a calibration attempt results are to meet a predetermined expectation. If not, then the calibration attempt would be considered as “fail” which means the results of the calibration would not be saved.

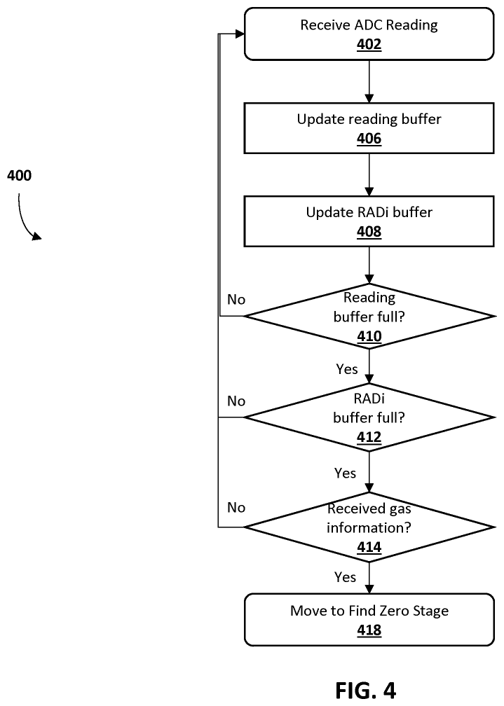

FIG. 4 illustrates, in a flowchart, an example of a method 400 of gathering the buffering data and receiving input, in accordance with some embodiments. The method 400 may be performed by the calibration unit 104 or an instrument 106 that includes logic performed by the calibration unit 104. The gas sensing instrument 106 is put into calibration mode, and begins receiving 402 the signal from the sensor 102 representing an analog-to-digital converter (ADC) reading. In some embodiments, the calibration mode involves having logic similar to that performed by the calibration unit 104 in the instrument 106. In other embodiments, the calibration mode may involve placing a device including the calibration unit 104 receiving readings from a gas sensor 102 associated with an instrument 106. The instrument 106 continues receiving data 402 which is passed to the calibration unit 104. The calibration unit 104 updates a reading buffer 406, which then also propagates to a buffer of the first differences in the reading 408. In some embodiments, a rolling average of the first differences (RADi) buffer is used. Once the reading buffer 410 and the RADi buffer 412 are full, the calibration unit 104 checks to see if it has received the required gas (or other target phenomena) information 414, which may include but is not limited to the gas concentration (or other physical quantity), for calibration, background gas (or other physical quantity), temperature, humidity and/or other factors which are known to affect (i.e., amplify, reduce or otherwise excite the signal). It should be noted that in some embodiments, the reading buffer and the RADi buffer may comprise one or more of the same or different buffers. If the buffers are full 410, 412 and the calibration unit 104 has received the required gas information 414, it 104 will move on to the next stage (which in this example is the optional Find Zero Stage 500 of FIG. 3 (or any initial starting point), but which could be the Waiting for Gas (or some other target phenomena or reading) 600 stage of FIG. 3 if some pre-determined zero were to be used). If the calibration were to be done with other hardware 108, the additional hardware 107 would need to receive gas readings for a predefined period, or until the additional hardware 108 tells the additional hardware 107 to stop receiving gas.

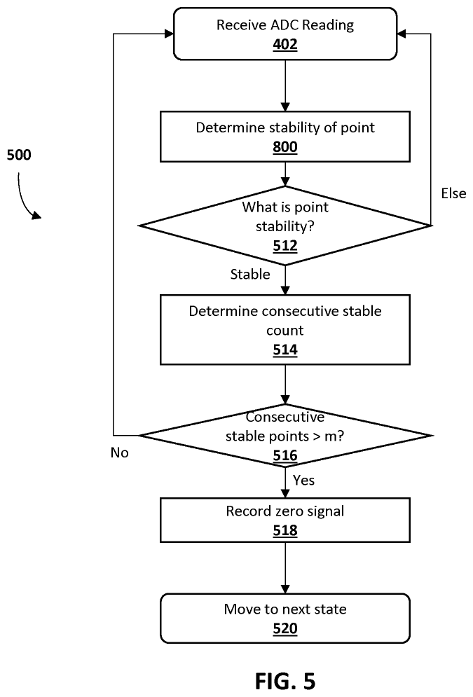

FIG. 5 illustrates, in a flowchart, an example of a method 500 of finding a zero, in accordance with some embodiments. The method 500 may be performed by the calibration unit 104 or an instrument 106 that includes logic performed by the calibration unit 104. The method 500 comprises the sensing instrument 106 (gas or other) which is put into calibration mode, and begins receiving 402 the signal from the sensor 102. The calibration unit 104 continues receiving data 402 and proceeds to determine the stability of the data 512 with zero gas (or any other starting gas value or other target phenomena) applied. If unstable, the calibration unit 104 will return to receiving the data 402 and continue until a stable point is read 512. If stable, the calibration unit 104 will count the consecutive stable observations 514, and proceed to determine if the consecutive stability is greater than a predefined threshold 516. If the threshold is passed, the zero signal is recorded 518 and the calibration unit 104 moves to the next state 520, otherwise the calibration unit 104 returns to the beginning of the process and receives the next observation 402. This threshold can be chosen based on predictions from a theoretical model or by experimentation. Increasing the threshold will decrease the likelihood of finding stabilization when the signal is still moving, but increases the expected time required for a stabilization to be found.

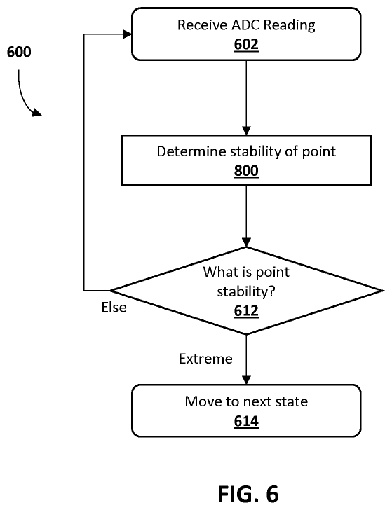

FIG. 6 illustrates, in a flowchart, an example of a method of waiting for gas (or other target phenomena) 600, in accordance with some embodiments. The method 600 may be performed by the calibration unit 104 or an instrument 106 that includes logic performed by the calibration unit 104. The method 600 comprises the sensing instrument 106 (gas or other) which is put into calibration mode, and begins receiving 402 the signal from the sensor 102. The instrument 100 continues receiving data 402 and proceeds to determine the stability of the data 512. If the data passes an extreme change in stability, the calibration unit 104 will recognize that gas is being applied and will move onto the next state 514, otherwise the calibration unit 104 returns to the beginning of the process and receives the next observation 402.

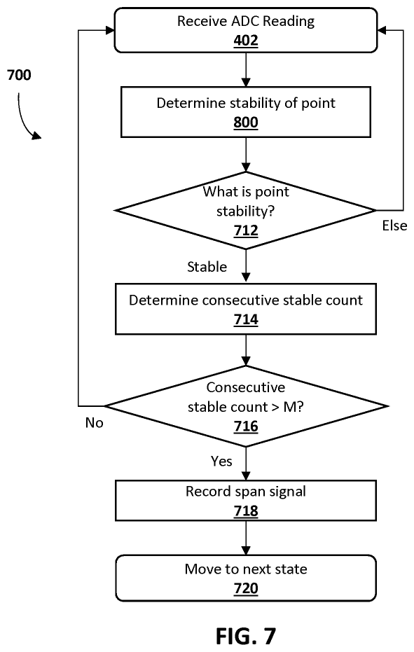

FIG. 7 illustrates, in a flowchart, an example of a method of determining the span 700, in accordance with some embodiments. The method 700 may be performed by the calibration unit 104 or an instrument 106 that includes logic performed by the calibration unit 104. The method 700 comprises the sensing instrument 106 (gas or other) which is put into calibration mode, and begins receiving 402 the signal from the sensor 102. The calibration unit 104 continues receiving data 402 and proceeds to determine the stability of the data 712 with a known concentration of gas (or other phenomena) applied. If unstable, the calibration unit 104 will return to receiving the data 402 and continue until a stable point is read 712. If stable, the calibration unit 104 will count the consecutive stable observations 714, and proceed to determine if the consecutive stability is greater than another predefined threshold 716. If this threshold is passed, the span signal is recorded 718 and the calibration unit 104 moves to the next state 720, otherwise the calibration unit 104 returns to the beginning of the process and receives the next observation 402. This threshold can be chosen based on predictions from a theoretical model or by experimentation. Increasing it will decrease the likelihood of finding stabilization when the signal is moving, but increases the expected time required for a stabilization to be found.

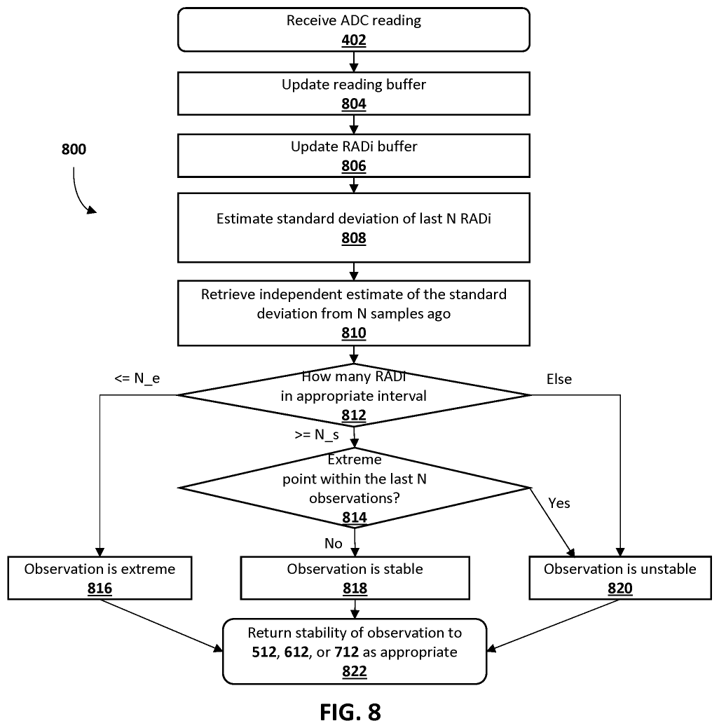

FIG. 8 illustrates, in a flowchart, an example of a method of determining stability 800, in accordance with some embodiments. The method 800 may be performed by the calibration unit 104 or an instrument 106 that includes logic performed by the calibration unit 104. The method 800 illustrates one embodiment of the detailed steps in determining stability to estimate the span and gain adjustment. The gain is adjusted using an estimator of the small random variations inherent in the signal. In some embodiments, this estimator is the sample standard deviation. In other embodiments, that gain may be adjusted, for example, by using a computational simplification of standard deviation estimation for minimizing processing and power requirements on the sensor board. In any case, once an estimator is chosen the stochastic properties of this estimator can be determined and analyzed using techniques familiar to those knowledgeable in the field.

The method 800 presents this latter embodiment, wherein twice the standard deviation of a set of samples is estimated using one half the difference between the maximum and minimum values of that. This method alleviates demand on hardware resources and allows a higher measurement precision when compared to calculating the sample standard deviation of a set of samples. However, this approach to estimating the standard deviation for gas (or other phenomena) sensing is readily extended to any estimator by using the small random variations inherent in the signal.

More specifically, this embodiment involves receiving the ADC reading 402 and storing it into the reading buffer 804. The reading is then used to populate the RADi buffer 806. Next the standard deviation estimator calculated N samples ago is retrieved 810 where N is the size of the RADi buffer. This is used to construct an interval where readings would be expected to be contained within if the signal were stable, and the number of entries in the RADi buffer contained in this interval is counted 812. It should be noted that if the value of two standard deviations is found to be less than the resolution value (the smallest increment that the instrument is capable of detecting or reporting in), then the standard deviation value is rounded up to so that the value of two standard deviations is the minimum resolution value. More specifically, in this example, if two standard deviations were to be less than one ADC count, the two standard deviation estimate would be rounded up to one ADC count. In one embodiment, sensor readings are classified into one of three classes based on what proportion of the RADi buffer falls within the interval constructed above. For brevity, we will call these classes stable, unstable, and extreme. The extreme classification would be expected when there is a rapid change in signal, such as immediately after gas (or other physical quantity) is applied to the sensor. The stable classification would be expected when the system has been allowed enough time to reach equilibrium. The unstable classification would be expected during the interim, where the change in the signal is neither rapid enough to be considered extreme nor slow enough to be considered stable.

In some embodiments we define N_s such that a reading is considered stable if a minimum of N_s samples in the RADi buffer are contained within the desired interval 818. Similarly we define a number N_e such that a reading is considered extreme if a maximum of N_e samples in the RADi buffer are contained within the desired interval 816. If neither of these conditions are met or additionally if the sample is not extreme but there has been an extreme sample in the last N samples 814, the reading is considered unstable 820. The parameters N_e and N_s can be chosen based on predictions from a theoretical model or by experimentation. Decreasing N_e will decrease the sensitivity to change in the signal before determining that a change in concentration has been observed. Increasing N_s will increase the specificity of the algorithm in determining when the signal has stabilized and consequently increase the expected time required for a stabilization region to be found. Finally, the stability classification of the sample is communicated to the instrument 822. It should be noted that the estimator may alternatively be performed using the range (rather than the range/4) or the sample standard deviation, etc.

For greater clarity, a common use case for gas sensing is described as follows. A parking garage will frequently have a CO detecting instrument in it. Common alarm levels might be 25 PPM (which would activate the HVAC system to dissipate or expel the gas) and 75 PPM (which would generate an audible and visual alarm to advise occupants. Upon initial factory calibration, a reading of 25 PPM might be associated with an ADC count of 2600. The sensor signal output often declines at a rate of 2% per month. Hence if an instrument was calculated in January, by June the instrument might only be reading 22 PPM when shown 25 PPM of gas. Consequently, a field service technician would indicate to the transmitter that they were going to begin calibration, expose the sensor to 25 PPM of gas, and wait until the instrument advised that a stabilization point had been found. For example, if N_e is 3 and N_s is 32, in the early part of the gassing, the slope would be steep and therefore there would be less than 3 samples in the RADi buffer that would be close enough to zero and therefore the reading would be determined to be extreme and the sample would not be stable. As gassing continued and the slope began to taper off, the number of samples that are in the RADi buffer that are close to zero would still be more than 3 but less than the threshold of 32. Eventually, more than 32 of the readings would be close enough to zero and therefore the sample would be deemed to be stable, and the corresponding ADC count would be recorded. If the corresponding ADC count was 2400, the firmware would then update the system's memory to reflect that 25 PPM of gas was associated with the 2400 ADC counts. When the sensor next encountered gas that caused it to reach the new ADC count recorded in memory, it would activate the HVAC system.

For illustrative purposes, the standard deviation estimator described above is compared against the more conventional sample standard deviation. The sample standard deviation of a set of observations {xᵢ=1 . . . N} is given by

$$√ ( \frac{\sum {( {x}_{i} - μ )}^{2}}{N - 1} ) where μ = \frac{1}{N} \sum {x}_{i} .$$

Thus, to calculate the sample standard deviation will require 2N addition operations, N multiplication operations, N subtraction operations, 2 division operations, a square root operation, and a final multiplication by 2, which can be simplified to a bitshift by 1. It should be understood that in some embodiments, a left shift will multiply and a right shift will divide. Typically, an addition, subtraction, or bitshift operation will take 1 clock cycle, integer multiplication will take 1-5, division will take 10-40, a square root will take 50-100, meaning a single calculation of a sample standard deviation will require up to 180+8N clock cycles, or for example about 430 clock cycles when 32 points are being considered at a time. In comparison, the calculating the aforementioned estimator of two times the standard deviation is done via the expression

$$\frac{max - min}{2}$$

where max and min are the maximum and minimum recorded observations, respectively. This requires only a subtraction followed by a division by 2, which can also be simplified to a bitshift by 1. Thus, a single calculation of this measure requires only about 2 clock cycles.

This simplification also carries benefits in terms of data loss from rounding errors. If each measurement is recorded with M bits of precision, then calculating the mean requires an additional log₂N bits. Squaring this number requires twice as many bits, and then another log₂N bits to add them up to calculate the variance. Combined, this means that a measurement with M bits will require a total of 2M+3 log₂N available bits plus an additional bit for sign or else information will be lost. In keeping with the example where N is 32, if 32 bits are available for computations, only 8 bits will be available for recording measurements. In addition, the final square root operation will lose 16 bits of information. In comparison, using the range to estimate the standard deviation means only M+2 bits are required to be available, meaning that in a 32-bit system 30 bits are available for recording measurements before loss of data occurs.

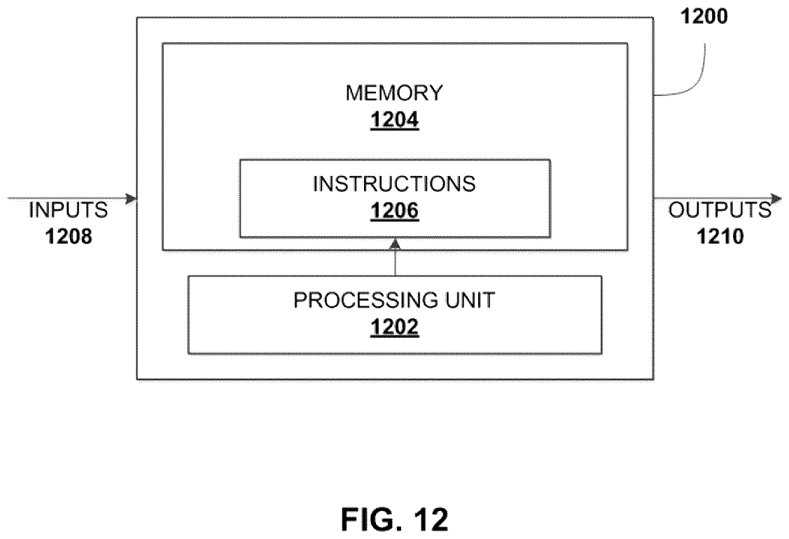

FIGS. 9A and 96 illustrate, in graphs 910 and 920, examples of sensor response versus time for prior methods using T80/90 (FIG. 9A) and Maximum Output (FIG. 9B). FIG. 9C illustrates, in a graph 930, an example of sensor response versus time for the method described herein, in accordance with some embodiments. As shown in the graphs 910, 920 and 930, the point at which the present disclosure determines a reading time 935 is more accurate than that for the T80/90 method 915 and faster than that for the Maximum Output method 925.

FIG. 9D illustrates, in a graph 940, the examples of sensor response versus time for all three methods of FIGS. 9A to 9C. FIG. 9D shows that readings for comparable time frames leading up to the T90 (915), “near maximum” (935) of the present disclosure, and the maximum (925). For the near maximum 935 (the present disclosure), the variation from the beginning of the period to the near maximum is smaller than the variation between successive readings within the range (see subgraph 944). Conversely, when looking at readings leading up to the T90 915, the variation between the beginning and end of over a similar time range is larger than the variation between successive readings within that time range (see subgraph 942). The variation over the period is also positive, but smaller than the variation between successive readings near the maximum output 925 (see subgraph 946). In the case of both the present disclosure near maximum 935 and the maximum 925, the response is still increasing, as it asymptotically approaches its absolute maximum, but the change over the time period is smaller than the change within the time period. Therefore, the present disclosure offers a method for identifying the first point in which the variation over a range is less than the variation within a range of successive, or near-successive samples by self-referencing the historical successive variations.

The example above illustrates the concept of using self-referencing historical variations to calibrate detection instrumentation. One skilled in the art would appreciate the similarities with the more complex RADical methodology used in the calibration of detection instrumentation described herein.

The embodiments of the disclosure described above offer a number of benefits, which can be illustrated through analysis of a series of examples. The examples below pertain to CO, NO2 and Oxygen, but relate more generally to any type of gas sensor. The above method is readily extended to new types of sensors (including non-gas sensors), so long as one is familiar with the small random variations inherent in the signal patterns inherent to that type of sensor.

Speed. As shown in a series of examples presented in Table 1 below, this calibration method returns output that is 96.3% to 98.6% of the near maximum output in usually less than one third of the time (e.g. 67.0 to 79.6 seconds for CO for the radical version, compared to between 222.1 and 235.8 seconds for full max). Table 1 shows an example of average statistics by calibration method.

TABLE 1
Average statistics by calibration method
Average Values

| | | | | | | Nitrogen | | | |
|---|---|---|---|---|---|---|---|---|---|
| | Carbon Monoxide | | | | | Dioxide | Oxygen | | |
| Serial Number | 719012A | 719012B | 719013A | 719013B | All | 719005A | 719017A | 719018A | All |
| Count of Gas.type | 17.0 | 20.0 | 14.0 | 14.0 | 65.0 | 33.0 | 8.0 | 10.0 | 18.0 |
| Output at zero gas | | | | | | | | | |
| sensor.response.min | 858 | 857 | 874 | 891 | 870 | 2,043 | 1,866 | 2,033 | 1,950 |
| radical.min | 868 | 874 | 884 | 900 | 881 | 2,041 | 1,868 | 2,036 | 1,953 |
| Sensor response at | | | | | | | | | |
| sensor.response.max | 2,934 | 2,979 | 2,834 | 2,922 | 2,909 | 1,505 | 2,611 | 2,624 | 2,617 |
| sensor.response.at.t80 | 2,519 | 2,554 | 2,442 | 2,516 | 2,501 | 2,554 | 2,462 | 2,506 | 2,484 |
| sensor.response.at.t90 | 2,726 | 2,767 | 2,638 | 2,719 | 2,705 | 1,559 | 2,537 | 2,565 | 2,551 |
| radical.max | 2,873 | 2,917 | 2,782 | 2,863 | 2,851 | 1,519 | 2,603 | 2,617 | 2,610 |
| radical.percent.of.max | 96.6 | 96.3 | 96.9 | 96.7 | 96.7 | 97.0 | 98.6 | 98.4 | 98.5 |
| Span | | | | | | | | | |
| sensor.response.span | 2,076 | 2,122 | 1,960 | 2,032 | 2,039 | −538 | 745 | 591 | 667 |
| radical.span | 2,005 | 2,044 | 1,898 | 1,963 | 1,970 | −522 | 734 | 581 | 657 |
| Seconds to | | | | | | | | | |
| time.to.max.s | 233.9 | 223.8 | 234.1 | 232.9 | 231.7 | 178.9 | 68.7 | 72.0 | 70.4 |
| t80.s | 14.7 | 13.0 | 13.8 | 12.5 | 13.6 | 3.4 | 2.9 | 2.5 | 2.7 |
| t90.s | 28.3 | 23.9 | 24.6 | 22.0 | 24.8 | 4.8 | 4.1 | 3.4 | 3.8 |
| radical.duration.s | 79.6 | 79.6 | 67.7 | 67.0 | 75.2 | 19.4 | 20.3 | 16.8 | 18.5 |

Accuracy. As shown in Table 2 below, this calibration method returns output that is more precise than the other methods.

TABLE 2
Measure of precision by calibration method
Coefficient of Variation (St. Dev/mean) - Measure of Precision

| | | | | | | Nitrogen | | | |
|---|---|---|---|---|---|---|---|---|---|
| | Carbon Monoxide | | | | | Dioxide | Oxygen | | |
| Serial Number | 719012A | 719012B | 719013A | 719013B | All | 719005A | 719017A | 719018A | All |
| Output at zero gas | | | | | | | | | |
| sensor.response.min | 6.4% | 3.8% | 7.4% | 7.0% | 6.6% | 2.9% | 0.4% | 0.4% | 4.3% |
| radical.min | 6.1% | 3.1% | 7.1% | 6.8% | 6.3% | 3.0% | 0.5% | 0.5% | 4.3% |
| Sensor response at | | | | | | | | | |
| sensor.response.max | 2.0% | 1.8% | 3.7% | 3.3% | 3.4% | 12.0% | 0.2% | 0.1% | 0.3% |
| sensor.response.at.t80 | 1.9% | 1.7% | 3.8% | 2.8% | 3.2% | 9.7% | 0.2% | 0.2% | 0.9% |
| sensor.response.at.t90 | 2.0% | 1.8% | 3.8% | 3.1% | 3.3% | 10.8% | 0.2% | 0.1% | 0.6% |
| radical.max | 1.9% | 1.4% | 3.9% | 2.6% | 3.2% | 11.6% | 0.2% | 0.1% | 0.3% |
| radical.percent.of.max | 1.5% | 1.7% | 1.4% | 1.6% | 1.5% | 0.7% | 0.6% | 1.1% | 0.9% |
| Span | | | | | | | | | |
| sensor.response.span | 3.8% | 3.3% | 4.0% | 7.2% | 5.6% | 23.6% | 1.3% | 1.7% | 11.7% |
| radical.span | 3.1% | 2.7% | 3.9% | 6.2% | 4.9% | 23.5% | 1.4% | 2.0% | 11.8% |
| Radical − Max | −0.7% | −0.6% | −0.1% | −1.1% | −0.6% | −0.2% | 0.0% | 0.3% | 0.1% |
| Seconds to | | | | | | | | | |
| time.to.max.s | 18.7% | 17.9% | 17.8% | 23.6% | 19.4% | 31.2% | 37.2% | 39.2% | 38.2% |
| t80.s | 27.8% | 32.1% | 27.0% | 34.9% | 30.2% | 32.8% | 31.0% | 24.9% | 29.1% |
| t90.s | 24.6% | 34.0% | 24.9% | 34.8% | 29.9% | 33.9% | 30.6% | 23.6% | 29.3% |
| radical.duration.s | 31.8% | 56.1% | 37.3% | 38.8% | 41.3% | 24.5% | 11.3% | 6.6% | 13.6% |

In general, the coefficient of variation of output response relative to maximum is typically lower for this method, than is the coefficient of variation (COV) of output for the other methods. This coefficient of variation for this faster version of gain estimation also produces a COV for the percent of maximum that ranges from 0.6% for Oxygen to a high of 1.7% for Carbon Monoxide.

In addition, because the system is self-referencing, it automatically adjusts the time for gassing based on temperature and humidity. This is evident from the COV's in respect to time to max, T80, T90 and the present disclosure. The COV for the time to maximum is highest for the present disclosure, suggesting it varies the most. This self-referencing adjustment for temperature and humidity is a key driver in the lower COV seen for the response for the present disclosure relative to other methods. In additional testing, inclusion of tests with more extreme temperatures and humidity ranges led to disproportionately higher COVs for the maximum sensor output than the “near maximum” sensor output derived from the method described in the present disclosure.

The estimation using the approximation of the standard deviation generates results that are just as precise as those generated from using the sample standard deviation, but achieved in a fraction of the computational time.

In another embodiment, the variations may be computed in a different way, while applying the same essential concept of the use of the small random variations in the signal relative to the change over a longer period. For example, the following algorithm has been applied to the calibration of an NO₂ sensor. It would find 97% of the maximum within 77 seconds, or 24% of the time that it would take to find the maximum. This algorithm would still have all the same beneficial aspects of the approach described above, in terms of being self-referencing, and self-adjusting in the length of time required to find a “near maximum”. This algorithm would offer the benefit of being computationally simpler than the one above, but the algorithm above found 97% of the maximum NO₂ signal in just 11% of the time during which gas was applied. Consequently, the use of the concept may be adjusted based on whether speed of response during calibration or minimization of computational power is most critical. For example, in the case of calibration of gas sensors, which involves the release of toxic, combustible and costly gases, speed may be more important.

Alternative algorithm applied to the calibration of an NO₂ sensor:

$${D}_{t} = {\sum }_{t = 1}^{16} {( {x}_{t} - {x}_{t - 1} )}^{2} - {( {x}_{t} - {x}_{t - 16} )}^{2}$$

If Σₜ₌₁¹⁶ Dₜ<0 for all D₁, . . . , D₁₆, then gassing (or other phenomenon) started.
If Σₜ₌₁¹⁶ Dₜ>0 for all D₁, . . . , D₁₆, then the near maximum has been reached.

TABLE 3
Time to Output response for alternative calculation equation for the
method of using variances as a basis of self-referencing calibration

| | | Time since start of |
|---|---|---|
| ADC counts | % of change in maximum response | gassing (seconds) |
| 2,278 | Not applicable - pre-response | 0 |
| 1,430 | 94.9% | 43 |
| 1,420 | 96.0% | 55 |
| 1,411 | 97.0% | 77 |
| 1,404 | 97.8% | 109 |
| 1,400 | 98.2% | 124 |
| 1,390 | 99.3% | 222 |
| 1,384 | 100.0% | 325 |

The algorithms discussed here represent just two examples and other embodiments of the principle of using variations of the signal in relation to the change in the signal over some period of time could also be developed.

As noted above, embodiments of the teachings herein may apply to the application of the calibration method to various types of sensors, such as, but not limited to, gas, temperature, humidity, vibration, pressure, motion, light, sound, particles, biosensors, etc. The target value each type of sensor may be a unit of measure typically used for the phenomena detected by that sensor. For example, the target value for a gas sensor may be a gas concentration level. For a temperature sensor, the target value is commonly a degrees centigrade level. For a humidity sensor, and the target value is commonly a relative humidity level. For a vibration sensor, the target value is commonly a rate of change in displacement per unit time level. For a pressure sensor, the target value is commonly a Pascal level. For a motion sensor or a light sensor, the target value is a frequency or luminosity. For a sound sensor, the target value is a change in sound pressure level (SPL). For particles, the target value is commonly the number of micrograms per cubic meter (μg/m3) parts per million. For microbes, the target value is commonly measure in cell numbers or cell mass. It should be noted that other units of measurements may be used for each sensor, and a person skilled in the art would understand which unit of measurement to use in different circumstances.

A further embodiment of this disclosure is that temperature and humidity correction factors can be derived from the shape of the curve itself.

FIG. 10 illustrates, in a schematic diagram, another example of a detection system 1000, in accordance with some embodiments. As shown in FIG. 10, the detection system 1000 comprises an instrument 106 comprising a first sensor 102 (e.g., a gas sensor or other type of sensor), a memory 1032, a processor 1034 and an input/output (I/O) unit 1036. Logic corresponding to the calibration unit 104 may be stored in the memory 1032 as firmware and/or software. The memory 1032, processor 1034 and I/O unit 1036 may be included on a system on chip 1030.

The instrument 106 may optionally include additional sensors 1022 such as a gas sensor, a temperature sensor, a humidity sensor, a pressure sensor, a vibration sensor, or other types of sensors, all of which may be used to contribute to the estimation of gas concentrations and/or gain adjustments for calibration. The instrument 106 may also optionally include at least one amplifier 1010 to amplify signals corresponding to gas readings such that the small “random” variations may better detected.

The system 1000 may optionally include additional sensors 1024 such as a gas sensor, a temperature sensor, a humidity sensor, a pressure sensor, a vibration sensor, or other types of sensors, all of which may be used to contribute to the estimation of phenomena measurements (e.g., gas concentrations) and/or gain adjustments for calibration. The system 1000 may also optionally include a Control or other system 1028, and an external device 1040 comprising its own memory 1042, processor 1044 and I/O unit 1046. The external device 1040 may comprise a smart phone, tablet, computer or other computing device that may be in communication with the instrument. For example, the external device 1040 may comprise logic corresponding to the calibration unit 104 such that the external device 1040 control the calibration of the instrument 106.

FIG. 11 is a schematic diagram of a computing device 1100 such as a server. As depicted, the computing device includes at least one processor 1102, memory 1104, at least one I/O interface 1106, and at least one network interface 1108.

Processor 1102 may be an Intel or AMD x86 or x64, PowerPC, ARM processor, or the like. Memory 1104 may include a suitable combination of computer memory that is located either internally or externally such as, for example, random-access memory (RAM), read-only memory (ROM), compact disc read-only memory (CDROM). The memory 1104 may store instructions corresponding to methods 200 to 800. The processor 1102 may execute the instructions.

Each I/O interface 1106 enables computing device 1100 to interconnect with one or more input devices, such as a keyboard, mouse, camera, touch screen and a microphone, or with one or more output devices such as a display screen and a speaker.

Each network interface 1108 enables computing device 1100 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others.

FIG. 12 shows a schematic representation of the calibration unit 104, as a combination of software and hardware components in a computing device 1200. The computing device 1200 may comprise one or more processing units 1202 and one or more computer-readable memories 1204 storing machine-readable instructions 1206 executable by the processing unit 1202 and configured to cause the processing unit 1202 to generate one or more outputs 1210 based on one or more inputs 1208. The inputs 1208 may comprise one or more signals representative of the inputs described in methods 200 to 800. The outputs 1210 may comprise one or more signals representative of the outputs described in methods 200 to 800.

Processing unit 1202 may comprise any suitable devices configured to cause a series of steps to be performed by computing device 1200 so as to implement a computer-implemented process such that instructions 1206, when executed by computing device 1200 or other programmable apparatus, may cause the functions/acts specified in methods 200 to 800 to be executed. Processing unit 1202 may comprise, for example, any type of general-purpose microprocessor or microcontroller, a digital signal processing (DSP) processor, an integrated circuit, a field programmable gate array (FPGA), a reconfigurable processor, other suitably programmed or programmable logic circuits, or any combination thereof.

Memory 1204 may comprise any suitable known or other machine-readable storage medium. Memory 1204 may comprise non-transitory computer readable storage medium such as, for example, but not limited to, an electronic, magnetic, optical, electromagnetic, infrared, or semiconductor system, apparatus, or device, or any suitable combination of the foregoing. Memory 1204 may include a suitable combination of any type of computer memory that is located either internally or externally to computing device 1200 such as, for example, random-access memory (RAM), read-only memory (ROM), compact disc read-only memory (CDROM) (or FLASH memory), electro-optical memory, magneto-optical memory, erasable programmable read-only memory (EPROM), and electrically-erasable programmable read-only memory (EEPROM), Ferroelectric RAM (FRAM) or the like. Memory 1204 may comprise any storage means (e.g. devices) suitable for retrievably storing machine-readable instructions 1206 executable by processing unit 1202.

The discussion provides example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

Although the embodiments have been described in detail, it should be understood that various changes, substitutions and alterations can be made herein.

Moreover, the scope of the present application is not intended to be limited to the particular embodiments of the process, machine, manufacture, composition of matter, means, methods and steps described in the specification.

As can be understood, the examples described above and illustrated are intended to be exemplary only.

What is claimed is:

1. A calibration system for calibrating an instrument, the calibration system comprising:
at least one sensor;
a processor; and
a memory comprising instructions which, when executed by the processor, configure the processor to:
obtain a series of sensor readings;
determine variations between changes in successive sensor readings from the series of sensor readings;
estimate a stabilization point of the sensor readings by identifying at least one sensor reading from the series of sensor readings at which the change in sensor reading from previous sensor readings is small relative to the variations; and
adjust, based on the stabilization point, a parameter in the instrument that represents an association between sensor readings and a known physical quantity.

2. The calibration system as claimed in claim 1, wherein the stabilization point comprises a point where the change in the sensor reading reaches near zero.

3. The calibration system as claimed in claim 1, wherein the processor is configured to:
update a reading buffer with the series of sensor readings;
populate a rolling average of the first differences (RADi) buffer based on the reading buffer data;
estimate a standard deviation of a last N sample RADi readings;
retrieve a standard deviation from a previous N sample of RADi readings prior to the last N RADi readings;
determine an interval where stable readings would fall; and
determine a count of RADi buffer entries in the interval.

4. The calibration system as claimed in claim 3, wherein the processor is configured to round up to a resolution value if the standard deviation from the previous N sample of RADi readings prior to the last N RADi readings is less than the resolution value.

5. The calibration system as claimed in claim 3, wherein the processor is configured to determine that the last N sample of RADi readings is stable when:
the count is greater than a first threshold value;
the count is greater than a second threshold value; and
there is no extreme RADi entry in the last N sample RADi readings.

6. The calibration system as claimed in claim 3, wherein the processor is configured to determine that the last N sample of RADi readings is unstable when:
the count is greater than a first threshold value;
the count is greater than a second threshold value; and
there is at least one extreme RADi entry in the last N sample RADi readings.

7. The calibration system as claimed in claim 3, wherein the processor is configured to determine that the last N sample of RADi readings is extreme when the count is less than or equal to a first threshold.

8. The calibration system as claimed in claim 1, wherein the processor is configured to:
associate a physical quantity with the sensor output as identified through the calibration process.

9. The calibration system as claimed in claim 1, wherein the processor is configured to:
associate a target value with the sensor output as identified through the calibration process.

10. A computer-implemented method of calibrating an instrument, the method comprising:
obtaining a series of sensor readings;
determining variations between changes in successive sensor readings from the series of sensor readings;
estimating a stabilization point of the sensor readings by identifying at least one sensor reading from the series of sensor readings at which the change in sensor reading from previous sensor readings is small relative to the variations; and
adjusting, based on the stabilization point, a parameter in the instrument that represents an association between sensor readings and a known physical quantity.

11. The method as claimed in claim 10, wherein the stabilization point comprises a point where the change in the sensor reading reaches near zero.

12. The method as claimed in claim 10, comprising:
updating a reading buffer with the series of sensor readings;
populating a RADi buffer based on the reading buffer data;
estimating a standard deviation of a last N sample RADi readings;
retrieving a standard deviation from a previous N sample of RADi readings prior to the last N RADi readings;
determining an interval where stable readings would fall; and
determining a count of RADi buffer entries in the interval.

13. The method as claimed in claim 12, comprising rounding up to a resolution value if the standard deviation from the previous N sample of RADi readings prior to the last N RADi readings is less than the resolution value.

14. The method as claimed in claim 12, comprising determining that the last N sample of RADi readings is stable when:
the count is greater than a first threshold value;
the count is greater than a second threshold value; and
there is no extreme RADi entry in the last N sample RADi readings.

15. The method as claimed in claim 12, comprising determining that the last N sample of RADi readings is unstable when:
the count is greater than a first threshold value;
the count is greater than a second threshold value; and
there is at least one extreme RADi entry in the last N sample RADi readings.

16. The method as claimed in claim 12, comprising determining that the last N sample of RADi readings is extreme when the count is less than or equal to a first threshold.

17. The method as claimed in claim 10, comprising:
associating a physical quantity value with the sensor output as identified through the calibration process.

18. The method as claimed in claim 10, comprising:
associating a target value with the sensor output as identified through the calibration process.

19. A calibration subsystem for calibrating an instrument, the calibration subsystem comprising:
a processor; and
a memory comprising instructions which, when executed by the processor, configure the processor to:
obtain a series of sensor readings from at least one sensor;
determine variations between changes in successive sensor readings from the series of sensor readings;
estimate a stabilization point of the sensor readings by identifying at least one sensor reading from the series of sensor readings at which the change in sensor reading from previous sensor readings is small relative to the variations; and
adjust, based on the stabilization point, a parameter in the instrument that represents an association between sensor readings and a known physical quantity.
